(19) United States
(12) Patent Application Publication
(10) Pub. No.: US 2005/0126562 A1
(43) Pub. Date: Jun. 16, 2005

(54) Treatment of breakthrough pain by drug aerosol inhalation

(75) Inventors: Joshua D. Rabinowitz, Princeton, NJ (US); William W. Shen, Stanford, CA (US); Martin J. Wensley, San Francisco, CA (US)

Correspondence Address: ALEXZA MOLECULAR DELIVERY CORPORATION, 1001 EAST MEADOW CIRCLE, PALO ALTO, CA, 94303, US

(73) Assignee: Alexza Molecular Delivery Corporation, Palo Alto, CA (US)

(21) Appl. No.: 10/996,162

(22) Filed: Nov. 23, 2004

Related U.S. Application Data
(60) Provisional application No. 60/530,058, filed on Dec. 15, 2003.

(51) Int. Cl. A61M 11/00

(52) U.S. Cl. 128/200.23; 128/200.14

(57) ABSTRACT

Devices comprising a housing defining an airway, at least one support configured to couple to the airway comprising at least one area selected from a first area and a second area, wherein a first compound is disposed on the first area, and a second compound is disposed on the second area, and wherein the second compound can counteract the pharmacological effects of the first compound; and a mechanism configured to release the first compound into the airway, wherein the device comprises at least one first area and at least one second area are disclosed. Methods of producing an aerosol of a first compound comprising providing at least one first area on which is disposed a first compound, and at least one second area on which is disposed a second compound, providing an airflow over at least a portion of the at least one first area, and releasing the first compound from at least a portion of at least one first area into the airflow, wherein the first compound forms an aerosol in the airflow, are also disclosed. Methods of using the devices and the methods of producing an aerosol are also disclosed.

### REFERENCE TO RELATED APPLICATIONS

This application claims priority to U.S. Provisional application Ser. No. 60/530,058 entitled “Treatment of Breakthrough Pain by Drug Aerosol Inhalation,” filed Dec. 16, 2003, Rabinowitz, Shen and Wensley, the entire disclosure of which is hereby incorporated by reference.

### BACKGROUND

1. Field

Embodiments generally relate to devices comprising a first compound and a second compound wherein the second compound can counteract the pharmacological effects of the first compound, and in particular, to devices for producing an aerosol of a first compound, to methods of producing an aerosol of a first compound using such devices, and to methods of using such devices and methods.

2. Introduction

Many potentially abusable drugs play an important role in current medical practice. Such abusable drugs include, for example, opioid analgesics, psycho-stimulants, cannabinoid agonists, dopamine agonists, steroids, and sedative hypnotics. For many abusable drugs, rapid, non-invasive delivery can have important medical advantages, including convenient fast onset of therapeutic effect, facilitation of patient titration to the minimum effective drug dose, dose reproducibility, and high bioavailability. Intrapulmonary administration of an aerosol comprising a potentially abusable drug is one means of effecting rapid drug delivery that can enable realization of the above benefits.

In administering an abusable drug to a patient, it can be advantageous to provide the drug in a form that mitigates the potential for misuse of the drug, either by the patient or by a drug abuser. Such misuse can take the form of excessive dosing of the drug by the intended route of administration, for example, by administering multiple doses instead of a single dose or by inhaling a nebulized drug solution for longer than the prescribed duration. Additionally, misuse can involve changing the route of administration of the drug, for example, by crushing a time-release capsule and then nasally ingesting the drug, or by intravenous injection of a drug solution intended for nebulization.

Electronic lockout means for preventing excessive use of an aerosol form of an abusable substance such as an opioid by its intended route of administration have been proposed. An example of the use of an electronic lockout feature to prevent an aerosol generating apparatus from producing aerosols more frequently than a prescribed time interval is disclosed in U.S. Pat. No. 5,694,919. While an electronic lockout feature can prevent overdosing, such an electronic lockout feature is ineffective at preventing misuse of the drug by changing the route of administration.

Providing abusable substances in a tamper-proof physical enclosure represents one method of preventing abuse by changing the route of administration. However, sequestering an abusable substance in a physical enclosure to prevent access by an abuser, as proposed in U.S. Pat. No. 5,694,919, can be difficult to implement in a manner that is both commercially viable and effective in protecting an abusable drug from misuse.

There is a need for improved devices and methods of preventing a drug formulation, and in particular a drug formulation comprising an abusable substance intended for aerosol delivery, from being extracted from the delivery apparatus for subsequent abuse.

### SUMMARY

A device is provided comprising a housing defining an airway, at least one support configured to couple to the airway comprising at least one area selected from a first area and a second area, wherein a first compound is disposed on the first area, and a second compound is disposed on the second area, and wherein the second compound can counteract the pharmacological effects of the first compound, and a mechanism configured to release the first compound into the airway, wherein the device comprises at least one first area and at least one second area. The potential for abuse of the first compound can be prevented or minimized by having the first compound and a second compound, which can counteract the pharmacological effects of the first compound, within the same device such that the first and second compounds are indistinguishable.

A method is provided for producing an aerosol of a first compound comprising providing at least one first area on which is disposed a first compound, and at least one second area on which is disposed a second compound, wherein the second compound can counteract the pharmacological effects of the first compound, providing an airflow over at least a portion of the at least one first area, and releasing the first compound from at least a portion of the at least one first area into the airflow, wherein the first compound forms an aerosol in the airflow.

It is to be understood that both the foregoing general description and the following detailed description are exemplary and explanatory only and are not restrictive of certain embodiments, as claimed.

### DESCRIPTION OF THE DRAWINGS

The accompanying figures, which are incorporated in and constitute a part of this specification, illustrate several embodiments and together with the description serve to explain certain embodiments.

FIG. 1 is a schematic illustration of a device consistent with certain embodiments.

FIG. 2 is a schematic illustration of a device consistent with certain embodiments.

FIG. 3 is a schematic illustration of a device consistent with certain embodiments.

FIG. 4 is a schematic illustration of a device consistent with certain embodiments.

FIG. 5A is a schematic illustration of a support consistent with certain embodiments.

FIG. 5B is a schematic illustration of a support consistent with certain embodiments.

FIG. 6A is a schematic illustration of a support consistent with certain embodiments.

FIG. 6B is a schematic illustration of a support consistent with certain embodiments.

FIG. 7A is a schematic illustration of a support comprising first and second areas consistent with certain embodiments.

FIG. 7B is a schematic illustration of a support comprising first and second areas consistent with certain embodiments.

FIG. 7C is a schematic illustration of a support comprising first and second areas consistent with certain embodiments.

FIG. 8 is a schematic illustration of a support consistent with certain embodiments.

FIG. 9 is a schematic illustration of control circuitry consistent with certain embodiments.

Unless otherwise indicated, all numbers expressing quantities of ingredients, reaction conditions, and so forth used in the specification and claims are to be understood as being modified in all instances by the term “about.”

In this application, the use of the singular includes the plural unless specifically stated otherwise. In this application, the use of “or” means “and/or” unless stated otherwise. Furthermore, the use of the term “including,” as well as other forms, such as “includes” and “included,” is not limiting. Also, terms such as “element” or “component” encompass both elements and components comprising one unit and elements and components that comprise more than one subunit unless specifically stated otherwise.

The section headings used herein are for organizational purposes only, and are not to be construed as limiting the subject matter described. All documents cited in this application, including, but not limited to patents, patent applications, articles, books, and treatises, are expressly incorporated by reference in their entirety for any purpose.

### DESCRIPTION OF VARIOUS EMBODIMENTS

Reference will now be made in detail to certain embodiments, examples of which are illustrated in the accompanying figures. Wherever possible, the same reference numbers will be used throughout the drawings to refer to the same or like parts.

In certain embodiments, a device comprises a housing defining an airway, at least one support configured to couple to the airway comprising at least one area selected from a first area and a second area, wherein a first compound is disposed on the first area, and a second compound is disposed on the second area, and wherein the second compound can counteract the pharmacological effects of the first compound, and a mechanism configured to release the first compound into the airway, wherein the device comprises at least one first area and at least one second area.

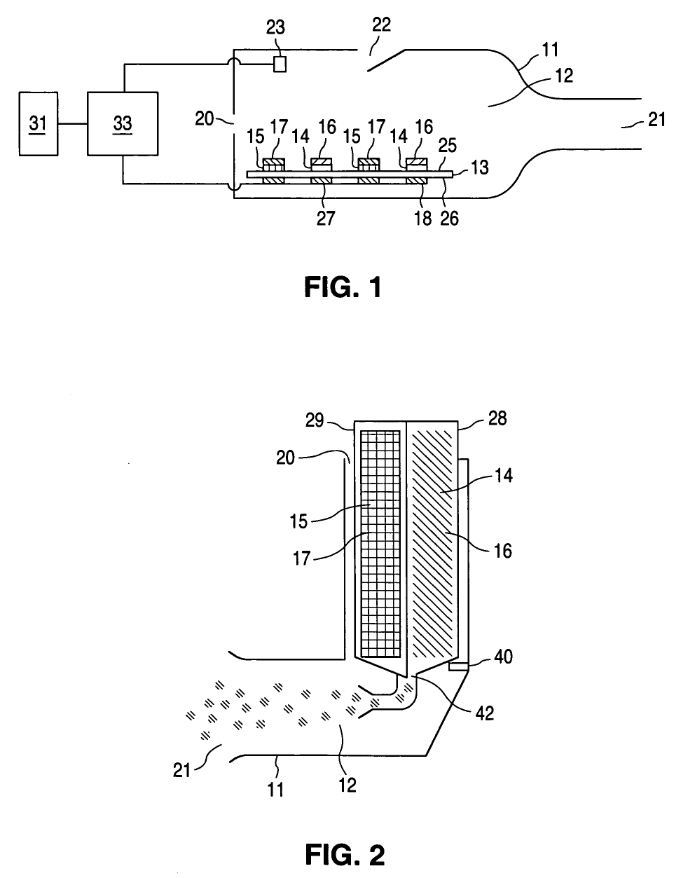

Certain embodiments of a device are schematically illustrated in FIG. 1. FIG. 1 shows embodiments of a portable inhalation device for the intrapulmonary delivery of an aerosol to a patient. The device shown in FIG. 1 can provide for multiple doses of a first compound, and each dose can be delivered to a patient in a single inspiration.

In certain embodiments, devices illustrated in FIG. 1 can comprise a housing 11 defining an airway 12. Airway 12 can include an inlet 20 and an outlet 21 to provide an airflow through airway 12, for example, upon inhalation through the mouth and/or the nostrils by a patient at outlet 21. In certain embodiments, the airflow rate and airflow velocity within airway 12 can be controlled by an airflow control valve 22 incorporated into the wall of housing 11. In certain embodiments, airflow control valve 22 can be a gate that allows additional air to enter airway according to the pressure differential between airway 12 and external to housing 11.

In certain embodiments, an actuation mechanism 23, capable of transducing the airflow velocity through airway 12 into an electrical or mechanical signal, such as, for example, a thermistor or pressure transducer, can be located in airway 12. In certain embodiments, actuation mechanism 23 can be electrically connected to a controller 33. Controller 33 can be further electrically connected to a power source 31 and to a release mechanism 18 comprising, for example, resistive heating elements. Controller 33 includes circuitry (not shown) to connect power source 31 to release mechanism 18 for controlling the release mechanism.

In certain embodiments, devices illustrated in FIG. 1 can include a support 13 disposed within airway 12. In certain embodiments, support 13 can comprise two first areas 14 comprising a first compound 16, and two second areas 15 comprising a second compound 17 disposed on a first surface 25 of support 13. In certain embodiments, first compound 16 and second compound 17 can be deposited as thin films on first areas 14 and second areas 15, respectively. In certain embodiments, release mechanism 18 can comprise resistive heating elements that can be located on or within a second surface 26 of support 13, opposing first areas 14 and second areas 15.

In certain embodiments, wherein electrical current provided by power source 31 is applied to resistive heating element 18, heat is generated. Heat generated by heating element 18 can be conducted through support 13, which, in certain embodiments, can comprise a thermally conductive material such as, for example, stainless steel, to heat at least one first area 14 and first compound 16 disposed thereon. When heated to a sufficient temperature, first compound 16 can thermally vaporize into airway 12, to form an aerosol comprising first compound 16 in the airflow within airway 12.

In certain embodiments, the operation of a device for intrapulmonary delivery of multiple doses of a first compound 16 according to FIG. 1 can be described as follows. A patient can inhale on outlet 21 of a device to generate an airflow through airway 12. Actuation mechanism 23, upon detecting a certain airflow velocity, can send a signal to controller 33. Upon receipt of the signal from actuation mechanism 23, controller 33 can electrically connect power source 31 to one of the resistive heating elements 18 underlying first area 14 comprising first compound 16. Heat generated by resistive heating element 18 can be conducted through support 13 to heat first area 14 causing first compound 16 to thermally vaporize to form an aerosol comprising agonist compound 16 in airway 12. The aerosol comprising first compound 16 can then be administered to the patient during inhalation, to deliver a dose of first compound 16 to the patient's respiratory tract. In certain embodiments, device activation and administration of a dose of first compound 16 can take place during a single inhalation.

In certain embodiments, for delivery of a subsequent dose of first compound 16, the same process can take place with the difference that controller 33 can connect a second resistive heating element 27 to power source 31. When activated by actuation mechanism 23 upon sensing a certain airflow velocity, second resistive heating element 27 can cause first compound 16 disposed on a second first area 14 to thermally vaporize and form an aerosol in the airflow through the airway 12. In certain embodiments, controller 33 can prevent the resistive heating elements from heating and releasing second compound 17 disposed on second areas 15.

In certain embodiments, misuse of first compound 16 can be minimized or prevented by locating second areas 15 comprising second compound 17 between first areas 14 comprising first compound 16 and/or by having the first compound 16 and second compound 17 be visually indistinguishable.

In certain embodiments, devices can be adapted to conventional aerosol delivery apparatus, such as, for example, metered-dose inhalers (MDIs), dry powder inhalers (DPIs), small volume nebulizers, large volume nebulizers, ultrasonic nebulizers, nasal sprays and the like. In certain embodiments, nebulizer inhalers can produce a stream of high velocity air that can cause a therapeutic agent to spray as a mist that can then be carried into a patient's respiratory tract. The therapeutic agent can be formulated in a liquid form such as a solution or a suspension of micronized particles typically exhibiting a diameter of less than 10 μm. In certain embodiments, DPIs can administer a therapeutic agent in the form of a free flowing powder that can be dispersed in a patient's air-stream during inspiration. A dry powder formulation can be loaded into a dry powder dispenser or into inhalation cartridges or capsules for use with a dry powder aerosol delivery device. In certain embodiments, MDIs can discharge a measured amount of a therapeutic agent using compressed propellant gas. Formulations for MDI administration can include a solution or suspension of an active ingredient in a liquefied propellant. The formulations can be loaded into an aerosol canister, which can form a portion of an MDI device.

Certain embodiments of devices adapted to an MDI format are schematically illustrated in FIG. 2. FIG. 2 illustrates an inhalation device comprising housing 11 that defines airway 12. Airway 12 includes inlet 20 and outlet 21 such that an airflow can be generated in airway 12 when a patient inhales on outlet 21 of the device either through the mouth or the nostrils. In other embodiments, inlet 20 is omitted, and gas flow is generated solely by release of pressurized material. In certain embodiments, airway 12 is omitted and aerosol is released directly from valve 42 into the environment. In certain embodiments, the device can comprise first area 14 comprising first compound 16, and second area 15 comprising second compound 17. In certain embodiments, first area 14 can be defined by support 28 in the form of a cartridge or canister and second area 15 can be defined by support 29 which also can be in the form of a cartridge or canister. First compound 16 and second compound 17 can be retained within the respective cartridges or canisters in the form of a solution or suspension comprising a liquefied propellant such as hydrofluoroalkane.

In certain embodiments, devices illustrated in FIG. 2 can be activated by a patient manually pushing cartridges 28 and 29 toward airway 12. When translated a certain distance toward airway 12, actuation mechanism 40 can open a release mechanism comprising, for example, a valve 42 allowing a propellant within first area 14 to inject a dose of liquid suspension comprising first compound 16 into airway 12 to form an aerosol comprising first compound 16 in the airflow. In certain embodiments, subsequent doses of first compound 16 can be released into airway 12 by repeated compression of cartridges 28 and 29 to reopen valve 42.

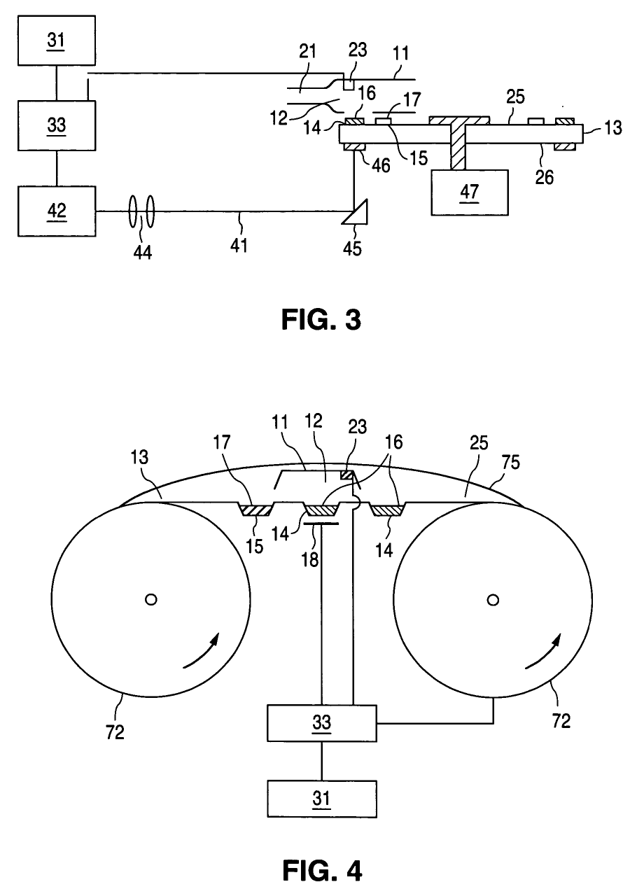

Certain embodiments of devices are schematically illustrated in FIG. 3. FIG. 3 illustrates certain embodiments in which support 13 can be in the form of a disk, and the release mechanism comprises optical heating. In certain embodiments, support 13 can comprise first area 14 comprising a thin film of first compound 16 and second area 15 comprising second compound 17. In certain embodiments, first area 14 and second area 15 can comprise stripes located on first surface 25 near the perimeter of support 13. In certain embodiments, first area 14 and second area 15 can comprise multiple first areas 14 and second areas 15 located on first surface 25 of support 13. In certain embodiments, the multiple first areas 14 and multiple second areas 15 can be interspersed. In certain embodiments, support 13 can be a thermally conductive material such as stainless steel.

In certain embodiments of devices illustrated in FIG. 3, for a certain rotational position of support 13, a portion of first area 14 and second area 15 can be coupled to airway 12 defined by housing 11. In certain embodiments, different portions of first area 14 and second area 15 can be coupled to airway 12 by rotating support disk 13 using a rotation mechanism 47. In certain embodiments, different first areas 14 and second areas 15 can be rotated into airway 12 by rotation mechanism 47. In certain embodiments, rotation mechanism 47 can comprise a manual and/or electronic advancement mechanism.

In certain embodiments, the heating release mechanism can comprise an optical source 42 to generate optical radiation 41 such as a Xenon flash lamp, an optical assembly that can include lenses 44 and reflectors 45 to direct and focus optical radiation 41 onto area 46 located on second surface 26 of support 13 underlying at least a portion of first area 14 comprising first compound 16 coupled to airway 12.

As an example of the operation of a device according to FIG. 3, a patient can inhale at outlet 21 of housing 11 to create an airflow in airway 12. At a certain airflow velocity, actuation mechanism 23 can send a signal to controller 33. Controller 33 can then connect power source 31 to optical source 42, to generate optical radiation 41. Optical radiation 41 can then be directed and focused onto area 46, causing local heating of support 13 underlying first area 14. Heat generated at area 46 of support 13 can then be conducted to a portion of first area 14, causing first compound 16 to thermally vaporize into the airflow to form an aerosol of first compound 16 in airway 12 which can then be inhaled by a patient. In certain embodiments, device activation and administration of first compound 16 can occur during a single inhalation by a patient.

In certain embodiments, subsequent doses of first compound 16 can be administered by advancing support 13 to couple a new portion of first area 14 and/or at least one new first area 14 to airway 12 and to optical radiation 41.

In certain embodiments, devices can be schematically illustrated in FIG. 4. FIG. 4 illustrates support 13 in the form of a tape having first areas 14 comprising first compound 16 and second areas 15 comprising second compound 17. In certain embodiments, support 13 can comprise, for example, a metal foil having recesses to contain first compound 16 and second compound 17. In certain embodiments, the recesses can facilitate retention of first compound 16 and second compound 17 such that first compound 16 and second compound 17 can be in the form of a dry powder, liquid, and/or thin film. In certain embodiments, support 13 can further comprise a protective layer 75 located on first surface 25 of support 13 on which first compound 16 and second compound 17 are disposed. In certain embodiments, protective layer 75 can comprise a polymer or metal film that can function to mechanically and/or environmentally protect the compounds, and, in certain embodiments, can be sealed to first surface 25 of support 13.

In certain embodiments, support 13 can be mechanically coupled to a reel-to-reel mechanism 72. In certain embodiments, advancing reel-to-reel mechanism 72 can move support 13 to couple a portion of support 13 comprising first area 14 on which is disposed first compound 16 to airway 12 defined by housing 11 and to release mechanism 18.

As an example of the operation of certain embodiments of a device illustrated in FIG. 4, reel-to-reel assembly 72 can advance support 13 to couple first area 14 comprising first compound 16 to airway 12, and to release mechanism 18. In certain embodiments, release mechanism 18 can comprise, for example, an ultrasonic source, a thermal source, or a source of electromagnetic radiation. During advancement, protective layer 75 can be removed from first surface 25 of support 13 to expose at least one dose of first compound 16.

A patient can inhale on the outlet of airway 12 (not shown) to generate an airflow in airway 12. In certain embodiments of devices illustrated in FIG. 4, when a certain airflow velocity is measured by actuation mechanism 23, a signal can be sent to controller 33. Controller 33 can electrically connect power source 31 to release mechanism 18 to release first compound 16 into airway 12 to form an aerosol comprising first compound 16. For example, in certain embodiments, release mechanism 18 can be an ultrasonic source that can produce an acoustic pulse that can eject first compound 16 from first area 14 into airway 12 to form an aerosol comprising first compound 16. In certain embodiments, an aerosol comprising first compound 16 can then be inhaled by a patient. In certain embodiments, following release of first compound 16 from first area 14, reel-to-reel mechanism 72 can advance support 13 to couple a second first area 14 to airway 12 and release mechanism 18. Upon actuation of release mechanism 18, a second dose of first compound 16 can be released into airway 12.

In certain embodiments, to prevent or minimize the potential for abuse of first compound 16, first areas 14 comprising first compound 16, and second areas 15 comprising second compound 17 can be randomly interspersed along the length of support 13. In certain embodiments, controller 33 can be programmed to advance reel-to-reel assembly 72 such that only first areas 14 comprising first compound 16 can be coupled to airway 12 and to release mechanism 18.

Certain embodiments of the invention are herein further described.

In certain embodiments, a housing can define the shape and dimensions of an airway, and can comprise at least one inlet, and at least one outlet. In certain embodiments, a housing can define more than one airway. In certain embodiments, a housing can be any appropriate shape or dimension for the intrapulmonary administration of an aerosol. In certain embodiments, a housing can have a shape and dimensions appropriate for portable use by a patient. “Patient” includes mammals and humans. In certain embodiments, a housing can be designed to accommodate and/or incorporate at least one support, an electronic controller, a release mechanism, an actuation sensor, a lock-out mechanism, as well as other components and/or features.

In certain embodiments, the dimensions of an airway can at least in part be determined by the volume of air that can be inhaled through the mouth or the nostrils by a patient in a single inhalation, the intended rate of airflow through the airway, and/or the intended airflow velocity at the surface of the support that is coupled to the airway and on which at least one first area is disposed. In certain embodiments, an airflow can be generated by a patient inhaling with the mouth on the outlet of the airway, and/or by inhaling with the nostrils on the outlet of the airway. In certain embodiments, an airflow can be generated by injecting air into the inlet such as for example, by mechanically compressing a flexible container filled with air and/or gas, or by releasing pressurized air and/or gas into the inlet of the airway. In certain embodiments, there is no airflow or airway and the device releases the aerosol into the environment directly, e.g., by passing a liquid under pressure through a valve or small holes.

In certain embodiments, a housing can be dimensioned to provide an airflow velocity through the airway sufficient to produce an aerosol of a first compound following release of the first compound from a first area into the airway. In certain embodiments, the airflow velocity can be at least 1 m/sec in the vicinity of the first area from which the first compound is released.

In certain embodiments, a housing can be dimensioned to provide a certain airflow rate through the airway. In certain embodiments, the airflow rate through the airway can range from 10 L/min to 120 L/min. In certain embodiments, the airflow rate can range from 10-60 L/min and, in other embodiments, from 10-40 L/min. In certain embodiments, an airflow rate ranging from 10 L/min to 120 L/min can be produced during inhalation by a patient when the outlet exhibits a cross-sectional area ranging from 0.1 cm² to 20 cm². In certain embodiments, the cross-sectional area of the outlet can range from 0.5 cm² to 5 cm², and in certain embodiments, from 1 cm² to 2 cm².

In certain embodiments, an airway can comprise one or more airflow control valves to control the airflow rate and airflow velocity in airway. In certain embodiments, an airflow control valve can comprise, but is not limited to, at least one valve such as an umbrella valve, a reed valve, a ball valve, a flapping valve that bends in response to a pressure differential, and the like. In certain embodiments, an airflow control valve can be located at the outlet of the airway, at the inlet of the airway, within the airway, and/or can be incorporated into the walls of housing defining the airway. In certain embodiments, an airflow control valve can be activated electronically such that a signal provided by a transducer located within the airway can control the position of the valve, or passively, such as, for example, by a pressure differential between the airway and the exterior of the device.

In certain embodiments, devices comprise at least one support, comprising at least one area selected from a first area and a second area. In certain embodiments, the support can retain a first compound, a second compound, or both a first compound and a second compound.

In certain embodiments, a support can comprise a release mechanism or certain elements of a release mechanism.

In certain embodiments, a support can comprise any appropriate shape and dimensions. Certain shapes for the support include, but are not limited to, rectangular inserts, cylindrical inserts, containers, cartridges, disks, tapes, and the like. In certain embodiments, a support can be a separate element or can be a surface of another element. For example, in certain embodiments, the support can be an inner wall of the housing, or can be the outer wall of the release mechanism, such as the outer wall of a heat package. In certain embodiments, a support can be an enclosure such as a container wherein the support defines an inner volume.

Certain embodiments of supports are schematically illustrated in FIGS. 1, 2, 3, and 4.

Certain embodiments of a support are schematically illustrated in FIG. 1. As shown in FIG. 1, support 13 can comprise a single structure in the form of a rectangular panel disposed within airway 12. In certain embodiments, support 13 can comprise two first areas 14 and two second areas 15 disposed on a first surface 25 of support 13, all of which are disposed within airway 12. First areas 14 and second areas 15 can be positionally distinguishable, meaning that the areas are discrete and do not overlap. In certain embodiments (not shown), first areas 14 and second areas 15 can be disposed on more than one surface of support 13. The support illustrated in FIG. 1, can comprise release mechanism 18 comprising resistive heating elements disposed on second surface 26 of support 13.

Certain embodiments of a support are schematically illustrated in FIG. 3. Support 13 can comprise a single support in the form of a disk comprising a first area 14 and a second area 15 disposed near the perimeter of the disk. In certain embodiments, only a portion of support 13 can be coupled to airway 12 and to release mechanism 41 for a particular rotational position of support 13. In certain embodiments, support 13 can be coupled to mechanism configured to move 47 such that support 13 can be rotated, or indexed, a certain amount to couple additional portions of first area 14 to airway 12 and to release mechanism 41.

Certain embodiments of a support are illustrated in FIG. 4 which include a single support 13 in the form of a tape comprising more than one first area 14. In certain embodiments, support 13 can be mechanically coupled to reel-to-reel assembly 72 such that support 13 can be advanced or indexed to couple at least one area 14 to airway 12 and to release mechanism 18.

In certain embodiments, devices illustrated in FIG. 2 can comprise a first support 28 comprising a first area 14 and a second support 29 comprising a second area 15. In certain embodiments, supports 28 and 29 can comprise a planar insert to define an area comprising first compound 16 and second compound 17, respectively. In certain embodiments, supports 28 and 29 can comprise a cartridge, canister or capsule to define a separate volume comprising first compound 16 and second compound 17, respectively.

In certain embodiments, a support can comprise a multilayer structure. For example, a support can comprise more than one layer of different materials to enable or facilitate the selective release of a first compound without releasing a second compound. The more than one layer comprising a support can extend over one or more surfaces of a support, or can be located in certain defined regions of a support. In certain embodiments, a first area on which a first compound is disposed, and a second area on which a second compound is disposed, can comprise more than one layer of differing compositions. The composition of the layers can be selected to facilitate the selective release of the first compound from the first areas without releasing the second compound from the second area.

In certain embodiments, the layers underlying a first and second area can have different thermal conduction properties. For example, a layer underlying a first area can be thermally conductive whereas a layer underlying a second area can comprise a thermal insulator. For such a structure, heat generated by a thermal release mechanism can more readily be conducted to the first compound thereby facilitating selective release of the first compound.

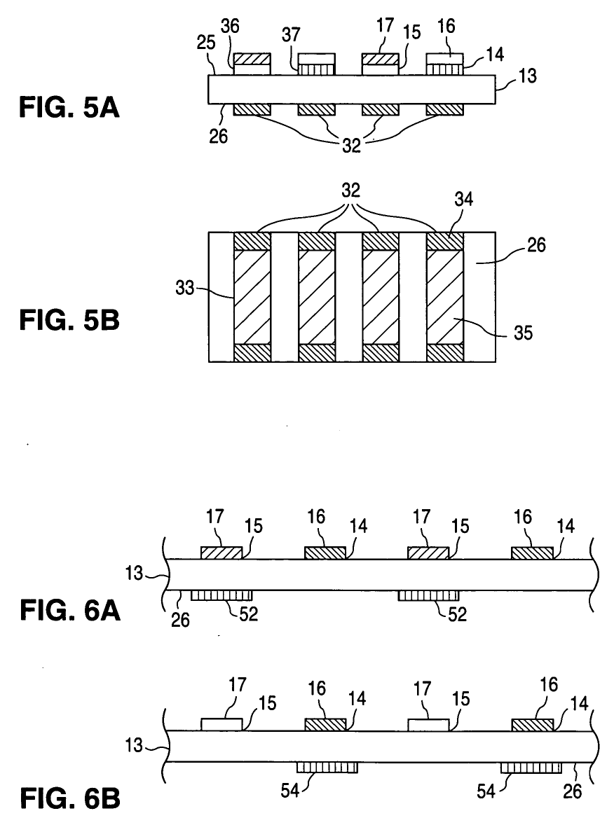

Certain embodiments of multilayer supports are illustrated in FIGS. 5A, 5B and 6. FIGS. 5A and 5B illustrate a multilayer support 13 which, in addition to a thin film of first compound 16, and a thin film of second compound 17, includes resistive heating elements 32, a thermally insulating layer 36 underlying second compound 17, and a thermally conducting layer 37 underlying first compound 16. In certain embodiments, resistive heating elements 32 can be located on second side 26 of support 13 and can underlie the first and second areas 15 disposed on first surface 25 of support 13. In certain embodiments, resistive heating elements 32 can include a layer of electrically resistive material such as carbon ink that produces heat when current is applied. In certain embodiments, resistive heating elements 32 can include electrical contact areas 34 to electrically connect the heating elements to control circuitry (not shown). In certain embodiments, when power is applied to resistive heating element underlying a first area 14 comprising first compound 16, the heat generated can be conducted to first compound 16 while minimizing heat conduction to second compound 17. Thermally insulating layer 36 can be, for example, a polymer or a ceramic. Thermally conducting layer 37 can be a metal such as, for example, copper, nickel, aluminum, or stainless steel.

In certain embodiments, the layers underlying a first and second area can have different electrical resistance properties. For example, a layer underlying a first area can have a high electrical resistance compared to that of the layer underlying a second area.

In certain embodiments, to facilitate selective release of a first compound, the first compound can be disposed on the surface of a first electrically conductive support, and the second compound can be disposed on the surface of a second electrically conductive support, wherein the electrical resistance of the first support is higher than that of the second support. Passage of the same amount of electrical current through the two supports can selectively heat the first support to selectively release the first compound. In certain embodiments, the first support can comprise, for example, stainless steel and the second support can comprise a metal having a lower resistivity such as copper or aluminum. In certain embodiments, the differential resistance can be created by using conductive supports, or conductive layers disposed on the supports, having different thickness. For example, in certain embodiments, the first compound can be disposed on a thin layer of gold or other electrically conductive material, disposed on a non-conductive support such as a ceramic. The second compound can be disposed on a layer of gold, or other electrically conductive material, that is thicker than the gold layer underlying the first compound, and which can also be disposed on a non-conductive support such as a ceramic. Current passing through both gold layers can differentially heat the thinner gold layer which exhibits a higher resistivity underlying the first compound than that of the thicker gold layer underlying the second compound, and thereby can selectively release the first compound from the support.

In certain embodiments, a layer can function as a protective cover disposed over a first compound and a second compound. In certain embodiments, the more than one layer can be a protective layer that can be removable to facilitate release of a first compound. In certain embodiments, a protective layer can comprise, for example, a metallic foil layer, plastic laminate layer, and the like. In certain embodiments, a protective layer formed from the more than one layer can be sealed to a support using adhesives, crimping, heat-sealing, and the like. In certain embodiments, a protective layer can protect the first compound from environmental degradation, or can mechanically protect the first compound from interaction with adjacent surfaces while packaged. In certain embodiments, a protective layer can be mechanically pulled from a support to expose the first compound immediately prior to coupling the first compound to the airway and release mechanism. Certain embodiments of supports having a protective layer are illustrated in FIG. 4.

In certain embodiments, a support can comprise a two-dimensional surface. In certain embodiments, a support can comprise recesses contiguous with the first areas in which the first compound is disposed. A recess can, for example, provide mechanical protection for a thin film of a first compound and/or can facilitate retention of a first compound in powder or liquid form. An example of a support having recesses is illustrated in FIG. 4.

In certain embodiments, a support can comprise slots, perforations or open areas which can be used, for example, for alignment, coupling to an advancement mechanism, or to thermally isolate a first area comprising a first compound from a second area comprising a second compound.

In certain embodiments, a support can comprise at least one area selected from a first area and a second area. Area, as used herein, refers to a positionally distinguishable region. In certain embodiments, an area can comprise a two-dimensional positionally distinguishable portion of a surface of a support. In certain embodiments, an area can comprise a three-dimensional positionally distinguishable volume defined by a support. Area can be used, for example, to refer to positionally distinguishable portions of a support, such as first area 14, and second area 15, as illustrated in FIG. 1, or a positionally distinguishable volume defined by a support such as a first area 14 and second area 15 defined by containers 28 and 29, respectively, as illustrated in FIG. 2.

In certain embodiments, a support can comprise a single first area, and in certain embodiments, more than one first area. In certain embodiments, a support can comprise a single second area and in certain embodiments, more than one second area. In certain embodiments, a support can comprise a single first area and a single second area; a single first area and more than one second area; more than one first area, and a single second area; or more than one first area and more than one second area.

In certain embodiments, a first area and a second area can comprise any appropriate shape and dimensions. The shape and dimensions of the first area and the second area disposed on one or more supports can be the same or different. In certain embodiments, the appropriate shape and dimensions of the first area and the second area can at least in part be determined by the shape and dimensions of the support on which the areas are disposed, the release mechanism employed in the device, the physical form of the first compound disposed on the first area, and/or the physical form of the second compound disposed on the first area. For example, when disposed on a two-dimensional surface, the first area and the second area can be in the shape of dots, squares, rectangles, circles, stripes, lines or exhibit an irregular shape. In certain embodiments, wherein the first area and the second area comprise a three-dimensional volume, the first areas and the second areas can take the shape of the enclosure defining the areas such as a cylinder or packet. In certain embodiments, the total number of first areas and the total number of the second areas comprising a device and/or a support can be the same or different, and the total surface area and/or volume of the first area and the second area comprising a device and/or support can be the same or different.

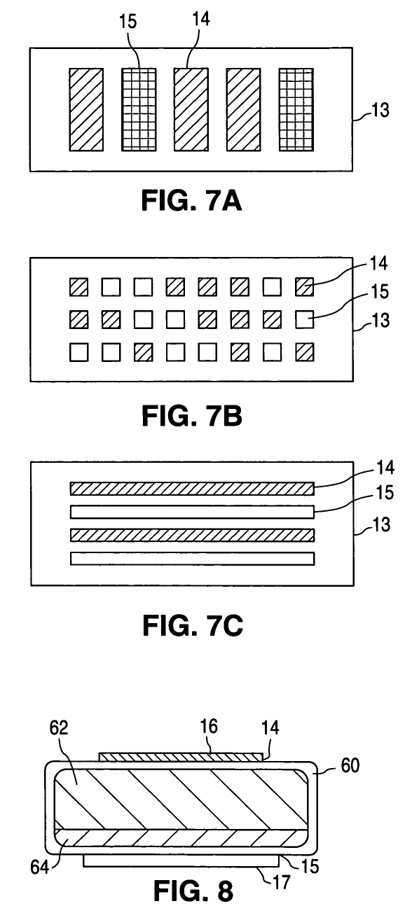

In certain embodiments, a first area and a second area can be positioned to complicate or prevent the selective removal of a first compound disposed on a first area other than by the release mechanism. Selective removal refers to the ability to remove a first compound disposed on a first area without removing a second compound disposed on a second area. For example, in certain embodiments, the first areas and the second areas can be interspersed. Examples wherein multiple first areas and multiple second areas are interspersed are schematically illustrated in FIGS. 7A, 7B, and 7C. As shown, FIG. 7A illustrates a row of interspersed first areas 14 and second areas 15 disposed on a surface of support 13. As shown, FIG. 7B illustrates an example in which multiple first areas 14 and multiple second areas 15 are irregularly interspersed. FIG. 7C illustrates an example of rows of interspersed first areas 14 and second areas 15 disposed on a surface of support 13.

In certain embodiments, complicating selective removal of a first compound from a first area can be realized by locating the first areas and the second areas in close spatial proximity. In certain embodiments, a neighboring first area and second area can be separated by less than 5 cm, in certain embodiments less than 2.5 cm, in certain embodiments less than 1 cm, in certain embodiments less than 0.5 cm, in certain embodiments less than 0.25 cm, and in certain embodiments less than 0.1 cm.

In certain embodiments, the minimum separation between a neighboring first area and second area can at least in part be determined by the minimum separation that can enable the selective release of the first compound from the first area without releasing the second compound from a second area. In certain embodiments, this can in part be determined by particular release mechanism employed in the device and the material composition of the support on which the first and second areas are disposed. For example, in certain embodiments in which a thermal release mechanism is used, the first compound and the second compound can be thermally isolated. In certain embodiments, thermal isolation can be accomplished, for example, not only by spatially separating the areas, but also by using multiple layers of materials with different thermal properties, as previously described. In certain embodiments, the support can also include physical features to thermally isolate the first compound and the second compound. For example, a support can include an opening located between neighboring first areas and second areas such that the support can be in the form of a web. In certain embodiments, a support can include a thermally insulating material located between the first and second areas. In certain embodiments, such as are schematically illustrated in FIG. 2, first compound 16 and second compound 17 can be retained in physically independent containers.

In certain embodiments, a first compound can be disposed on a first area. A first compound refers to a chemical substance such as a drug. In certain embodiments, the first compound is a drug capable of combining with a cell receptor and initiating a reaction or activity typically produced by the binding of an endogenous substance.

In certain embodiments, a first compound can be disposed on a first area in any physical form capable of being released from a first area by a release mechanism with minimal degradation, reaction or modification of the first compound. An appropriate physical form of a first compound disposed on a first area can at least in part be determined by the release mechanism employed in a particular embodiment. For example, a first compound disposed on a first area can comprise a solid thin film, a powder, a particulate, or a liquid. In certain embodiments, wherein a first compound comprises a powder, the particles comprising a first compound can exhibit a diameter ranging from 0.1 μm to 100 μm. In certain embodiments, wherein a first compound comprises a solid thin film, the thickness of the thin film can be less than 30 μm, in certain embodiments less than 20 μm, and in certain embodiments less than 100 μm. The appropriate thickness of a thin film can at least in part be determined by the film thickness at which the first compound can be released into an airway with minimal degradation or reaction. For example, an appropriate film thickness using a thermal vaporization release mechanism can be less than 10 μm and greater than 0.01 μm.

In certain embodiments, a first compound can comprise any appropriate chemical form, which can at least in part be determined by the particular release mechanism employed in a specific embodiment, and to minimize degradation, reaction or modification of the first compound, for example during storage or release from the first area. In certain embodiments, such as for example, where a first compound is dissolved or suspended in a liquid, the first compound can be in the form of a salt of the first compound. In certain embodiments where a first compound is disposed on a first area as a solid thin film, the first compound can be in pure form (e.g., freebase or free acid form), and in certain embodiments, the first compound can be crystalline or it can be amorphous.

In certain embodiments, a first compound can comprise a pharmaceutically acceptable compound. “Pharmaceutically acceptable” refers to approved or approvable by a regulatory agency of the Federal or a state government or listed in the U.S Pharmacopoeia or other generally recognized pharmacopoeia for use in animals, and more particularly in humans. Since intrapulmonary administration can rapidly introduce a pharmaceutical compound into the systemic circulation of a patient, a first compound can be a pharmaceutical compound in which rapid onset of treatment is indicated.

An example of one such class of pharmaceutical compounds in which rapid onset of treatment is indicated is opioid analgesics for the treatment of pain. Opioid analgesics can be used in the treatment of postoperative pain, cancer pain, back pain, headache pain and most other forms of moderate to severe pain. Thus, in certain embodiments, a first compound can comprise an opioid analgesic, such as for example, fentanyl, sufentanyl, remifentanyl, morphine, hydromorphone, oxymorphone, codeine, hydrocodone, oxycodone, meperidine, methadone, nalbuphine, buprenorphine, and buorphanol.

Another class of pharmaceutical compounds useful in certain embodiments include a sedative hypnotic, such as benzodiazepines, for the treatment of acute panic attacks, acute anxiety, and sleep induction. In certain embodiments, a first compound can comprise a non-benzodiazepine sedative hypnotic, including, for example, propofol, chloral hydrate, zaleplon, zolpidem, zopiclone, indiplon, pentobarbital, and other barbiturates. In certain embodiments, a first compound can comprise a benzodiazepine sedative hypnotic, including, for example, chlordiazepoxide, clonazepam, clorazepate, diazepam, flurazepam, estazolam, lorazepam, temazepam, alprazolam, oxazepam, and triazolam.

Another class of pharmaceutical compounds useful in certain embodiments include cannabinoid agonists for the treatment of, for example, anorexia, nausea, vomiting, multiple sclerosis, and pain. In certain embodiments, a first compound can comprise a cannabinoid agonist, such as, for example, dronabinol, and cannabidiol.

Another class of pharmaceutical compounds useful in certain embodiments include dopamine agonists for the treatment of, for example, Parkinson's disease and depression. In certain embodiments, a first compound can comprise a dopamine agonist such as, for example, bromocriptine, levodopa, pergolde, pramipexole, ropinirole, and selegiline.

Another class of pharmaceutical compounds useful in certain embodiments include stimulants for the treatment of, for example, attention deficit hyperactivity disorder, promotion of alertness, and depression. In certain embodiments, a first compound can comprise a stimulant such as, for example, amphetamine, methylphenidate, modafinil, phentermine, and sibutramine.

Another class of pharmaceutical compounds useful in certain embodiments include steroids for the treatment of for example hormonal imbalances and breast cancer. In certain embodiments, a first compound can comprise a steroid such as for example testosterone, precursors of testosterone, release enhancers, and pharmacological mimics.

In certain embodiments, a first compound can comprise a single pharmaceutical compound or can comprise a combination of more than one pharmaceutical compound. In certain embodiments, a first compound can comprise a pharmaceutical composition comprising a first compound, and a pharmaceutically acceptable carrier. In certain embodiments, the carrier can comprise, for example, solvents, excipients, and/or particulates. In certain embodiments, such carriers can include those generally recognized as appropriate for pharmaceutical compositions as found, for example in Remington: The Science and Practice of Pharmacy, 20^{th} Edition, Lippincott Williams & Wilkins, Philadelphia, Pa., 2000.

In certain embodiments, a composition comprising a first compound can comprise substances to enhance release, aerosol formation, intrapulmonary delivery, therapeutic efficacy, therapeutic potency, stability, and the like. For example, to enhance therapeutic efficacy a first compound can be coadministered with an active agent to increase the absorption or diffusion of the first compound through the pulmonary alveoli, or to inhibit degradation of the first compound in the systemic circulation. In certain embodiments, the first compound can be in the form of a salt to enhance chemical stability in a liquid solvent. In certain embodiments, the first compound can be in an uncomplexed form to facilitate release by thermal vaporization. In certain embodiments, the first compound can be co-administered with active agents having pharmacological effects that enhance the therapeutic efficacy of the first compound. In certain embodiments, a first compound can comprise compounds that can be used in the treatment of one or more diseases, conditions, or disorders.

In certain embodiments, a first compound can comprise an abusable substance. “Abusable substance” refers to a substance that can be improperly used, for example, by administering more than a prescribed or intended dosage, or by altering the route of administration from the intended route. For example, an opioid analgesic can be abused by using the opioid analgesic to elicit a euphoric effect, rather than therapeutically for the treatment of pain. Abusable substances include substances regulated by a regulatory agency focused on preventing drug abuse, such as, for example, the United States Drug Enforcement Agency (DEA). In certain embodiments, an abusable substance can be a substance listed on DEA schedule II, III, IV, or V.

In certain embodiments, a second compound can be disposed on a second area. A second compound is a chemical compound that can act to reduce or to counteract the physiological activity and/or pharmacological effects of another chemical substance and/or brings about an effect, including, for example, but not limitation, nausea, headache, etc. that reduces the desire to abuse another chemical substance. In certain embodiments, a second compound can counteract the physiological activity or pharmacological effects of an endogenous or exogenous chemical substance. Endogenous chemical substance refers to relating to or produced by metabolic synthesis in the body or system. Exogenous chemical substance refers to introduced from or produced outside the body or system. An example of an exogenous chemical substance is a first or second compound administered to a patient. In certain embodiments, a second compound can be a compound that reduces or counteracts the physiological activity and/or pharmacological effects of a first compound and/or reduces the desire to abuse the first compound.

In certain embodiments, a second compound can comprise a pharmaceutically acceptable compound. In certain embodiments, a second compound can be selected from at least one chemical substance that counteracts the pharmacological effects of a first compound disposed on a first area. For example, in certain embodiments wherein the first compound comprises an opioid analgesic, the second compound can comprise an antagonist of an opioid analgesic, for example, naloxone or naltrexone. In certain embodiments, wherein the first compound comprises the opioid analgesic fentanyl, the second compound can comprise at least one fentanyl antagonist compound, such as, for example, naloxone or naltrexone.

In certain embodiments, wherein the first compound comprises a sedative hypnotic, the second compound can comprise an antagonist of a sedative hypnotic, such as, for example, flumazenil.

In certain embodiments, wherein the first compound comprises a cannabinoid agonist, the second compound can comprise an antagonist of a cannabinoid agonist, such as, for example, SRI 41716 (rimonabant).

In certain embodiments, wherein the first compound comprises a dopamine agonist, the second compound can comprise an antagonist of a dopamine agonist, such as, for example, clozapine, olanzapine, quetiapine, risperidone, ziprasidone, fluphenazine, haloperidol, perphenazine, pimozide, thiothixene, trifluoperazine, loxapine, molidone, prochlorperazine, chlorpromazine, mesoridazine, and trioridazine.

In certain embodiments, wherein the first compound comprises a stimulant, the second compound can comprise an antagonist of a stimulant.

In certain embodiments, wherein the first compound comprises a steroid, the second compound can comprise an antagonist of a steroid.

A second compound can comprise a single pharmaceutical compound or a combination of more than one pharmaceutical compound. In certain embodiments, a second compound can comprise a pharmaceutical composition comprising the second compound, and a pharmaceutically acceptable carrier. In certain embodiments, the carrier can comprise, for example, solvents, excipients, and/or particulates. In certain embodiments, a second compound can comprise substances to inhibit release by the release mechanism, therapeutic efficacy, therapeutic potency, stability, and the like, as previously described. In certain embodiments, a second compound can further comprise compounds capable of counteracting the pharmacological effects of one or more first compounds.

To prevent or minimize the potential for abuse of a first compound by selectively removing the first compound other than by the release mechanism, the first compound disposed on a first area, and the second compound disposed on a second area can exhibit certain similar physical properties. In certain embodiments, the first compound disposed on a first area can be visually indistinguishable from the second compound disposed on a second area. For example, in certain embodiments, wherein the first compound is in the form of a powder, the second compound can also comprise a powder. Similarly, in certain embodiments wherein the first compound comprises a thin film, the second compound can also comprise a thin film having a thickness similar to that of the thickness of the thin film comprising the first compound. Other examples of visual characteristics that can be matched include, for example, color and texture. In certain embodiments, a first compound disposed on a first area and a second compound disposed on a second area can exhibit similar physical characteristics. For example, a first compound and a second compound can be soluble to the same or a similar degree in the same solvents, and/or can exhibit the same or similar melting points. In certain embodiments, the compounds can exhibit the same or similar average particle size or the same or similar viscosity. Similar physical and visual characteristics for the first compound and the second compound can minimize the ability of abuse of the first compound by complicating the ability of an abuser to identify and/or distinguish the two compounds.

In certain embodiments, a first compound and a second compound can be applied to a first area and a second area, respectively, by any appropriate method. In certain embodiments, the compounds can be applied to the respective areas as a solution or suspension in a liquefied propellant. In certain embodiments, the compounds can be applied as a free flowing powder comprising micronized particles. In certain embodiments, the compounds can be applied to the first and second areas by thin film deposition techniques, such as inkjet printing, spray coating, electrostatic coating, dip coating, and the like. In certain embodiments, the methods and materials used to apply the compounds to the respective areas can maintain the pharmaceutical acceptability and therapeutic efficacy of the compounds.

In certain embodiments, devices can provide for single-dosing or multi-dosing capability. A dose refers to the amount of first compound released during a single activation of the device. In certain embodiments, the amount of first compound released can be similar to the amount of first compound administered to a patient.

In certain embodiments, the dose of a first compound released can represent a therapeutically effective amount of a first compound. “Therapeutically effective amount” refers to the amount of a compound that, when administered to a patient for treating a disease, condition or disorder, is sufficient to affect such treatment of the disease, condition or disorder. The “therapeutically effective amount” can vary depending on the compound, the disease, condition or disorder and its severity and the age and weight of the patient to be treated. “Treating” or “treatment” of any disease, condition, or disorder refers to arresting or ameliorating a disease, condition, symptom, or disorder, reducing the risk of acquiring a disease, condition or disorder, reducing the development of a disease, condition, disorder or at least one of the clinical symptoms of the disease, condition or disorder, or reducing the risk of developing a disease, condition or disorder or at least one of the clinical symptoms of a disease or disorder. “Treating” or “treatment” also refers to inhibiting the disease, condition, symptom, or disorder, either physically, e.g. stabilization of a discernible symptom, physiologically, e.g., stabilization of a physical parameter, or both, and inhibit at least one physical parameter which may not be discernible to the patient. Further, “treating” or “treatment” refers to delaying the onset of the disease, condition, symptom, or disorder or at least symptoms thereof in a patient which may be exposed to or predisposed to a disease, condition or disorder even though that patient does not yet experience or display symptoms of the disease, condition or disorder.

The amount of first compound administered can be determined by a physician in the light of relevant circumstances, including the disease, condition or disorder to be treated, the actual compound administered, the age, weight, and response of the individual patient, the severity of the patient's symptoms, and the like. In certain embodiments, the dose of a first compound to be administered can be determined by the patient using a device.

In certain embodiments, a first compound can be administered in doses at periodic weekly, daily, or hourly intervals, or intermittently, as appropriate. In certain embodiments, a treatment regimen can comprise administration over extended periods of time ranging from weeks to months, or the treatment regimen can comprise chronic administration.

Since a therapeutically effective amount of a first compound can vary depending on a number of factors, in certain embodiments, a dose can comprise a fixed or pre-defined amount of a first compound. For example, in certain embodiments, activating a device can release first compound from only a single first area wherein the amount of first compound disposed on a single first area comprises a dose. In certain embodiments, activating a device can release a first compound from two first areas, and in certain embodiments, more than two first areas, each release corresponding to an individual dose of a first compound.

In certain embodiments, a dose can be pre-set and in certain embodiments, can be controlled by a patient to enable the patient, for example, to establish and/or maintain a therapeutically effective amount of a first compound comprising a dose throughout the course of a treatment regimen. In certain embodiments, the amount of a first compound released during a single activation, e.g. a dose, can be a fixed or pre-defined amount. However, appreciating that a therapeutically effective amount of a first compound can vary depending on a number of factors, the amount of first compound released during a single activation can be adjustable. In certain embodiments wherein the dose can be adjustable, an upper limit to the amount of first compound released during a single actuation can be established to prevent abuse by overdosing.

In certain embodiments, dose titration can be accomplished by adjusting the amount of first compound released. The amount of a first compound released can be titrated, for example, by controlling the number of positionally distinguishable first areas from which a first compound is released, or by controlling the amount of first compound released from a first area. For example, in some embodiments comprising a thermal vaporization release mechanism, different amounts of a first compound comprising the aerosol can be provided by releasing the first compound from a different number of first areas. In certain embodiments, a dose of a first compound can be adjusted by controlling the temperature applied to the first area, or by adjusting the surface area of first compound which is heated and thereby control the amount of the first compound released. In certain embodiments, the ability to adjust the dose of the first compound can be appropriate in the treatment of diseases, conditions, or disorders, such as pain, which manifest acute onset of symptoms of variable intensity.

In certain embodiments, a device can provide for a multiple dosing capability. A multiple dosing capability can enable certain devices to deliver multiple doses of a first compound as required for the treatment of a disease, condition, symptom, or disorder. For the treatment of certain conditions such as acute onset pain, it is anticipated, for example, that a treatment regimen can comprise from 5 to 15, from 2 to 50, or from 1 to 100 doses of an opioid analgesic per day. An exemplary therapeutically effective amount of opioid analgesic can comprise 5 mg, which can be deposited to cover a surface area of 15 cm². Thus, multiple doses of a first compound can be provided in a small surface area. In certain embodiments, each positionally distinguishable first area comprising a first compound can represent an individual dose and in certain embodiments, more than one positionally distinguishable first area can represent a dose of first compound. In certain embodiments, a single first area can comprise multiple doses, with only a portion of the first compound being released into the airway during each successive activation of the device.

In certain embodiments, multiple dosing devices can include a mechanism to advance or index the first areas that can be activated to release a first compound into an airway. Advancing or indexing can comprise electronic mechanisms, mechanical mechanisms, or a combination of electronic and mechanical mechanisms. For example, with reference to FIG. 1, each first area 14 comprising first compound 16 can represent a single dose. Following release of first compound 16 from one first area, for example, by resistive heating, controller 33 can enable a subsequent dose to be released by electrically connecting another heating element to release a first compound from a second first area 14, and so forth. A subsequent dose of first compound 16 can then be released into airway 12. As another example, with reference to FIG. 4, following release of first compound 16 from first area 14, support 13 can be advanced by reel-to-reel mechanism 72 to couple a new first area 14 to airway 12. First compound 16 on new first area 14 can then be released into airway 12 by release mechanism 18 during a subsequent activation of the device.

In certain embodiments, providing multiple doses can include releasing additional first compound from a single first area. In certain embodiments, such as when the first compound is in the form of a liquid or powder following each dose, additional doses of the first compound can be coupled to the airway by a valve as shown in FIG. 2.

In certain embodiments, a release mechanism can comprise, for example, a mechanical mechanism, a thermal mechanism, or an acoustic mechanism. Examples of mechanical release mechanisms include valves such as are used in nebulizer inhalers, dry powder inhalers, and metered-dose inhalers which can release a solution and/or suspension of a first compound, or micronized particles comprising a first compound into an airflow. In certain embodiments, the release mechanism can comprise one or more pistons that can inject a formulation through a nozzle or an array of holes to produce an aerosol. In certain embodiments, mechanical release mechanisms can include mechanisms for removing a protective layer such as a polymer film or metal foil to expose a solution and/or suspension of a first compound, or a dispersion and/or powder of micronized particles comprising a first compound to an airflow.

In certain embodiments, a release mechanism can comprise an acoustic mechanism. For example, ultrasonic pulses can be used to inject a solution and/or suspension of micronized particles or a powder comprising a first compound disposed on a first area into the airflow.

In certain embodiments, a release mechanism can comprise a thermal mechanism such that a first compound disposed on a first area in the form of a solid thin film can be thermally vaporized to release a first compound into an airflow. To thermally vaporize a first compound, a number of mechanisms for imparting thermal energy to a first area disposed on a support can be employed. In certain embodiments, thermal release mechanisms include, for example, resistive heating, optical heating, and chemical heating.

Certain embodiments in which a thermal release mechanism comprises resistive heating are schematically illustrated in FIG. 5. FIG. 5 illustrates a support 13 having a plurality of first areas 14 and a plurality second areas 15, disposed on a first surface 25 of support 13. A plurality of resistive heating elements 32 can be disposed on a second surface 26 of support 13. The plurality of resistive heating elements 32 can be positioned such as to be opposed to first areas 14 and second areas 15, as shown. Resistive heating elements 32 can comprise an ohmic material 35, for example graphite ink, such that heat is generated when an electrical current is applied to the resistive heating element 32. Heat generated by resistive heating element 32 can be conducted through support 13 to first compound 16 disposed on first area 14. In certain embodiments, support 13 can comprise a thermally conductive material such as a metal, for example, stainless steel, copper, nickel or aluminum. In certain embodiments, support 13 can comprise devices and/or features for electrically connecting resistive heating elements 32 to control circuitry 33 and power source 31. In certain embodiments, support 13 can comprise electrical contacts 34 or electrical connectors (not shown) electrically connected to the resistive heating elements 32.

In certain embodiments, a thermal release mechanism can comprise generating heat by means of the absorption of electromagnetic energy. Electromagnetic energy includes for example infrared, microwave, radiofrequency, and visible portions of the electromagnetic spectrum. An electromagnetic thermal release mechanism can comprise a power source, an electromagnetic energy source, and a lens assembly for transmitting and coupling the electromagnetic energy to heat a first compound. In certain embodiments, the electromagnetic energy source can comprise an optical source such as a Xenon flash lamp, laser diode, light emitting diode, and the like.

Certain embodiments of devices comprising an optical source for heating the first compound are schematically illustrated in FIG. 3. Certain embodiments of devices illustrated in FIG. 3 include a power source 31 electrically connected to controller 33 and electromagnetic source 42. Electromagnetic energy, for example, optical radiation generated by electromagnetic source 42 can be transmitted through an optical assembly comprising, for example, lenses 44 and reflectors 45 to focus optical radiation 41 onto support 13. In certain embodiments, optical radiation 41 can impinge on an area 46 disposed on second surface 26 of support 13, opposing first area 14 disposed on first surface 25 of support 13. Heat generated at area 46 by absorption of incident radiation 41 can be conducted through thermally conductive support 13 to thermally vaporize first compound 16, releasing first compound 16 into airway 12.

In certain embodiments, area 46 can comprise a layer of material exhibiting optical properties which facilitate the selective release a first compound 16. For example, as shown in FIG. 6A, layer 52 on second surface 26 of support 13 can include an optically reflective material opposing second areas 15 on which is disposed second compound 17. Layer 52 can reflect incident radiation to cause differential heating of first areas 14 and second areas 15, thereby facilitating selective release of first compound 16.

In certain embodiments, as shown in FIG. 6B, the region of second surface 26 of support 13 opposing first areas 14 can include areas 54 comprising a material which facilitates generation of thermal energy. For example, areas 54 can comprise a material capable of absorbing the incident electromagnetic radiation, such as, carbon black for the absorption of optical radiation. Alternatively, in certain embodiments, areas 54 can comprise an antireflective coating that can transmit incident electromagnetic radiation to support 13 underlying first areas 14 while other areas of second surface 26 can be reflective.

In certain embodiments, the electromagnetic radiation can be incident directly on a first compound. Heat generated by the absorption of the incident radiation by the first compound can thermally vaporize the first compound. In such embodiments, the temporal and power of the incident electromagnetic radiation can be selected to minimize degradation of the first compound.

In certain embodiments, a thermal release mechanism can comprise a chemical heat source. Examples of chemical heat sources include exothermic electrochemical reactions and metal oxidation reactions. As an example of a metal oxidation reaction, heat can be generated by igniting a fuel mix comprising a metal such as zirconium, titanium, iron or magnesium, and an oxidizer such as molybdenum trioxide, potassium perchlorate, potassium chlorate, Teflon, boron, or Iron (III) oxide, in combination with a binder such as nitrocellulose, polyvinyl alcohol or a colloidal dispersion of silicon dioxide.

In certain embodiments, a chemical heat source can be contained within a heat package such that the outer expanse of the heat package can comprise a thermally conductive material such as stainless steel. The outer expanse of the heat package can comprise the support on which at least one area selected from a first area and a second area can be disposed. In such embodiments, differential heating of the first area so as to release only the first compound can be accomplished, for example, by having first and second areas, or the region of the support underlying the first and second areas, exhibit thermal properties that facilitate the release of the first compound from the first areas while inhibiting release of the second compound from the second areas. For example, as schematically illustrated in FIG. 8, heat package 60 can comprise a thermally insulating layer 64 located between chemical heat source 62 and the second area 15 on which is disposed second compound 17. Insulating layer 64 can comprise, for example, a ceramic, or an air gap. First compound 16 can be disposed on first area 14. To facilitate selective release of first compound 16, the region underlying first area 14 does not include a layer of thermally insulating material.

In certain embodiments, a thermal vaporization release mechanism can release a first compound from the first area with minimal degradation, reaction, or modification of the first compound to produce an aerosol comprising the first compound.

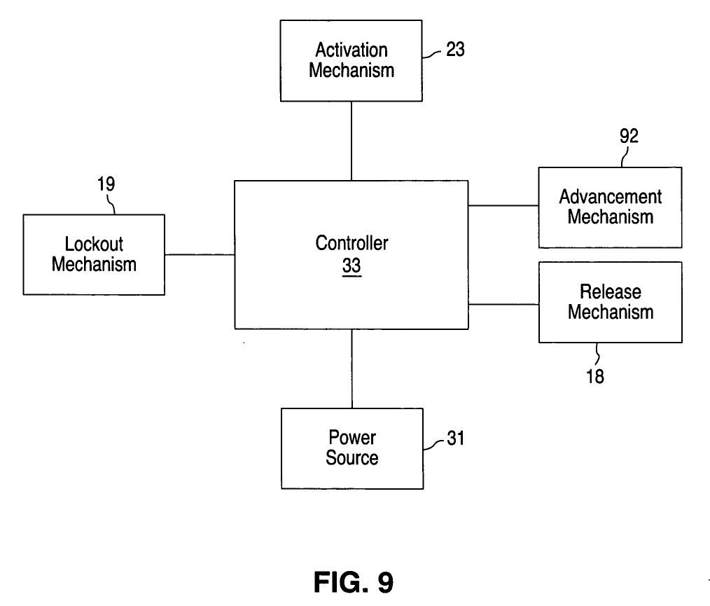

In certain embodiments, devices can comprise electronic control circuitry. Electronic control circuitry can control activation of the device, and in certain embodiments having multiple dosing capability, the time between doses. Certain embodiments of control circuitry are schematically illustrated in FIG. 9. FIG. 9 illustrates a power source 31, activation mechanism 23, a lockout mechanism 19, and release mechanism 18, coupled to controller 33. In certain embodiments, controller 33 can control the activation of release mechanism 18. In certain embodiments, activation of release mechanism 18 can be determined by actuation mechanism 23 and lockout mechanism 19. Actuation mechanism 23 can send a signal to controller 33 when a certain airflow velocity is detected in airway 12 of the device. Lockout mechanism 19 can include timing circuitry (not shown) that can send a signal to controller 33 after a certain timer period has elapsed. Upon receipt of signals form both actuation mechanism 23 and lockout mechanism 19, controller 33 can enable release mechanism 18.

In certain embodiments of devices having multiple dosing capability, a control signal generated by controller 33 can activate mechanism configured to move 92 also be used to advance or index a support to couple at least one new first area or a new portion of a first area to airway 12 and to the release mechanism 18. In certain embodiments, controller 33 can couple a new release mechanism 18 to a new first area 14. For example, controller 33 can connect a different resistive heating element to power source 31. In certain embodiments, enabling release mechanism 18 can include electrically connecting power source 31 to the release mechanism 18, or in embodiments having a mechanical release mechanism, can disengage a mechanical stop.

In certain embodiments wherein the device can be for portable use, control circuitry can be incorporated within the housing of the device. In certain embodiments, certain of the subsystems of the control circuitry can be located within the device, and other subsystems can be located external to device. For example, the power source, controller, and lockout mechanism can be external to housing, while the release mechanism and the actuation mechanism can be located within the housing of the device. For portable operation, the power source can comprise primary cells such as disposable batteries or secondary cells such as rechargeable batteries. In certain embodiments, wherein certain of the subsystems can be located external to the housing, the electronic control circuitry can comprise a means, such as a cable, for electrically connecting the external and internal subsystems.

In certain embodiments, the airway can include an actuation mechanism. An actuation mechanism can be positioned within the airway and can be coupled to the controller. An actuation mechanism can activate the control circuitry when a certain airflow velocity is detected in the airway. In certain embodiments, when the actuation mechanism is not activated, the controller can prevent activation of the release mechanism, for example by preventing electrical connection between the power source and the release mechanism, thereby preventing release of the first compound. The airflow velocity at which the actuation mechanism is activated can be set to a pre-established threshold airflow velocity. In certain embodiments, the pre-established threshold can be at least 1 m/sec. The pre-established threshold airflow velocity can be set to ensure that an aerosol comprising the first compound is formed following release of the first compound from a first area. In certain embodiments, an actuation mechanism can be any appropriate sensor capable of transducing or converting airflow velocity in the airway into an electrical or mechanical signal, such as, for example, a thermistor or pressure transducer.

In certain embodiments, an actuation mechanism can comprise a mechanical switch. For example, as schematically illustrated in FIG. 2, a mechanical switch 40 can be located within housing 11 of a device such that when cartridges 28 and 29 are manually translated toward airway 12, a valve can be opened to release a first compound 16 into the airway 12.

In certain embodiments, and in particular those embodiments comprising a multiple dosing capability, a controller can comprise a lockout mechanism. A purpose of a lockout mechanism can be to prevent abuse or minimize the potential to abuse the first compound by repeated dosing. In certain embodiments, a lockout mechanism can prevent reactivation of the release mechanism for a certain time period following a prior activation. In certain embodiments, a lockout mechanism can comprise timing circuitry. Control and timing circuitry can be implemented using a microcontroller or a combination of analog and digital logic. In certain embodiments, following delivery of a dose, the control circuitry can disable a switch to prevent electrical connection of a power source to a release mechanism thereby preventing release of the first compound. After a certain time period, as determined, for example, by timing circuitry, the controller can activate the switch to connect the power source to the release mechanism thereby enabling release of a first compound. In certain embodiments, the delay period can be minutes, hours or days. In certain embodiments, the appropriate time between repeated activations of the device can at least in part be determined by the severity and manifestations of the disease, condition, or disorder to be treated, the potency of the pharmaceutical compound being administered, the duration of the therapeutic effect of the pharmaceutical compound being administered, the physiological condition of the patient, and the like. In certain embodiments, the timing cycle of the lockout mechanism can be set when manufactured, or by a physician directing treatment.

In certain embodiments, a lockout mechanism can impose a simple, fixed duration of time between repeated deliveries of a first compound. In such embodiments, the duration of time between repeated deliveries of a first compound can be 1, 3, 5, 10, 15, 20, 30, 45, or 60 minutes, in certain embodiments can be 1.5, 2, 3, 4, 6, 8, 12, or 24 hours, and in certain embodiments, can range from 2 to 7 days. In certain embodiments, wherein a first compound comprises an opioid agonist, the lockout interval can range, for example, from 3 to 60 minutes. In certain embodiments, the lockout mechanism can allow delivery of a fixed number of doses of the first compound within a certain time period, such as, for example, 3 doses per 30 minutes, or 8 doses per day, without control of the time interval between individual successive doses.

In certain embodiments, the lockout mechanism can control both the time interval between successive doses, and the number of doses within a certain time period. For example, in certain embodiments wherein the first compound comprises the opioid analgesic, fentanyl, a lockout strategy can impose a time interval ranging from 2 to 6 minutes between successive doses, and prevent delivery of more than from 2 to 8 doses within a time period ranging from 30 minutes to 4 hours. In certain embodiments, wherein for example, the first compound comprises fentanyl, the lockout strategy can impose a time interval of 4 minutes between successive doses, and prevent delivery of more than 4 does per hour. In certain embodiments, a lockout mechanism can impose a fixed time interval between successive doses, a maximum fixed number of doses per one fixed time interval, and a greater maximum fixed number of doses per longer fixed time interval. For example, in certain embodiments, a lockout mechanism can impose a fixed time interval of 3 minutes between successive doses, with a maximum of 3 doses within a 30 minute period, and a maximum of 24 doses within a 24 hour period.

In certain embodiments, during release of a first compound, at least part of a support comprising a first area on which the first compound is disposed can be coupled to the airway. Coupling a support to an airway can comprise inserting all, or a part of the support into the airway. In certain embodiments, a support can be located adjacent to an airway such that a release mechanism can inject a first compound into the airway. In such embodiments, the support can be coupled to the airway through openings in a housing or through openings in a support. In certain embodiments, the openings in the housing can include valves. For example, as shown in FIG. 1, support 13 can be completely disposed within airway 12, or as shown in FIG. 2, the support comprising containers 28 and 29 can be disposed adjacent to airway 12 and coupled to airway 12 through valve 42.

In certain embodiments, a support can be moved to couple additional first areas or other portions of a first area to the airway and/or the release mechanism. Such embodiments can include a mechanism to move the support, for example, as illustrated in FIG. 3 and FIG. 4 wherein the mechanism configured to move comprises a rotation apparatus and a reel-to-reel assembly, respectively.

In certain embodiments, devices can comprise a mechanism to generate or augment the airflow rate through and/or airflow velocity within the airway. Mechanisms for generating or augmenting the airflow rate through and/or airflow velocity in the airway can include, for example, pressurized gas sources, and compressible containers.

Certain embodiments include methods of producing an aerosol of a first compound comprising providing at least one first area on which is disposed a first compound and at least one second area on which is disposed a second compound, wherein the second compound can counteract the pharmacological effects of the first compound, providing an airflow over at least a portion of the at least one first area, and releasing the first compound from at least a portion of the at least one first area into the airflow; wherein the first compound forms an aerosol in the airflow.

Certain embodiments include methods of administering a therapeutically effective amount of a first compound to a patient comprising inhaling an aerosol produced by the devices and methods of producing an aerosol.

Certain embodiments include devices and methods of treating a disease in a patient in need of such treatment comprising administering to the patient an aerosol comprising a therapeutically effective amount of at least one first compound, wherein the aerosol is produced by devices and methods of producing an aerosol. By enabling administration of at least one pharmaceutical compound to the respiratory tract of a patient, devices and methods of producing an aerosol can be suited for the treatment of diseases, conditions, or disorders in which rapid therapeutic effectiveness is advantageous, such as for example, asthma, anaphylaxis, pain, acute panic attacks, acute anxiety, sleep induction, and nausea, vomiting, Parkinson's disease, depression, and attention deficit hyperactivity disorder.

In certain embodiments, devices and methods produce aerosols of a first compound for intrapulmonary delivery and rapid absorption of the first compound into the systemic circulation. Following release of a first compound into the airway of a device, the first compound can combine with the airflow to form an aerosol comprising the first compound. In certain embodiments, depending in part on the form of the first compound and the particular release mechanism employed, the first compound can be injected into the airflow as a particulate, as liquid droplets or as a vapor.

For administration of a first compound, the dimensions of the particulates of the first compound comprising the aerosol can be within a range appropriate for intrapulmonary delivery. Particles having a mass median aerodynamic diameter (“MMAD”) ranging from 1 μm to 3 μm, and ranging from 0.01 μm to 0.10 μm are recognized as particularly appropriate for intrapulmonary delivery of pharmaceutical compounds. Aerosol particles characterized by a MMAD ranging from 1 μm to 3 μm can deposit on alveoli walls through gravitational settling and can be absorbed into the systemic circulation, while aerosol particles characterized by a MMAD ranging from 0.01 μm to 0.1 μm can also absorbed through alveoli walls by diffusion. Particles characterized by a MMAD in the range between 0.10 μm to 1 μm are frequently exhaled. Thus, in certain embodiments, aerosols produced using devices and methods of producing an aerosol can comprise particles having a MMAD ranging from 0.01 μm to 5 μm, in certain embodiments, a MMAD ranging from 0.05 μm to 3 μm, and in certain embodiments, a MMAD ranging from 1 μm to 3 μm. In certain embodiments, aerosols suitable for intrapulmonary delivery of pharmaceutical compounds can further be characterized by the geometric standard deviation of the log-normal particle size distribution. In certain embodiments, aerosols produced using the devices and methods of producing an aerosol comprise a geometric standard deviation of the log-normal particle size distribution of less than 3, in certain embodiments, less than 2.5, and in certain embodiments, less than 2.

In certain embodiments, factors such as the airflow velocity, the release mechanism and the physical form of a first compound disposed on the first area can be selected to produce an aerosol comprising particles characterized by a MMAD ranging from 1 μm to 5 μm, and in certain embodiments ranging from 0.01 μm to 0.1 μm. For example, by heating a thin film of a first compound having a thickness of less than 10 μm to a temperature ranging from 200° C. to 600° C. within less than 500 msec to thermally vaporize the first compound into an airflow having a velocity of at least 1 m/sec can produce an aerosol comprising particles characterized by a MMAD in a range appropriate for intrapulmonary administration of the first compound.

A further characteristic of an aerosol for intrapulmonary delivery of a pharmaceutical compound is the purity of the pharmaceutical compound comprising the aerosol. In certain embodiments, an aerosol can comprise predominantly a first compound and ambient air. Under certain conditions, a first compound can degrade, react or otherwise be modified during application, during storage and transportation, or during release. In certain embodiments, aerosols formed using the devices and methods of producing an aerosol comprise greater than 90% by mass a first compound, and in other embodiments greater than 95% by mass a first compound. In certain embodiments, less than 10% by mass of a first compound released to form an aerosol is degraded, reacted or modified during release from first area, and in other embodiments, less than 5% by mass of a first compound released to form an aerosol is degraded, reacted or modified during release from first area.

In certain embodiments, thermal vaporization conditions previously described can also produce an aerosol in which less than 10% by mass of the first compound is degraded during release, and in certain embodiments, less than 5% by mass of the first compound is degraded during release.

Certain embodiments include an aerosol comprising a first compound produced by the devices and methods of producing an aerosol. In certain embodiments, the aerosol can comprise more than one first compound and can comprise additional pharmaceutically acceptable compounds.

Other embodiments will be apparent to those skilled in the art from consideration of the specification and practice of the devices and methods consistent with embodiments of the invention as disclosed herein. It is intended that the specification and examples be considered as exemplary only, with a true scope and spirit of the devices and methods consistent with embodiments of the invention being indicated by the following claims.

1. A device comprising:
a housing defining an airway;
at least one support configured to couple to the airway comprising at least one area selected from a first area and a second area, wherein a first compound is disposed on the first area, and a second compound is disposed on the second area, and wherein the second compound can counteract the pharmacological effects of the first compound; and
a mechanism configured to release the first compound into the airway coupled to the support;
wherein the device comprises at least one first area and at least one second area.

2. The device of claim 1, wherein the mechanism to release does not release the second compound into the airway.

3. The device of claim 1, wherein an aerosol comprising the first compound is formed when the first compound is released into the airway.

4. The device of claim 1, wherein the device comprises more than one airway.

5. The device of claim 1, wherein the support comprises more than one support.

6. The device of claim 1, wherein the support comprises more than one first area.

7. The device of claim 1 wherein the support comprises more than one second area.

8. The device of claim 1, wherein the support comprises more than one first area and more than one second area.

9. The device of claim 8, wherein the more than one first areas are spatially interspersed among the more than one second areas.

10. The device of claim 1, wherein the at least one first area and the at least one second area are disposed on different supports.

11. The device of claim 1, wherein the at least one first area and the at least one second area are disposed on the same support.

12. The device of claim 1, wherein the first compound disposed on the first area and the second compound disposed on the second area are indistinguishable to visual observation.

13. The device of claim 1, wherein the at least one first area and the at least one second area are positionally distinguishable.

14. The device of claim 1, wherein the mechanism configured to release releases the first compound from a single first area into the airway.

15. The device of claim 1, wherein the mechanism configured to release releases the first compound from more than one first area into the airway.

16. The device of claim 1, wherein the mechanism configured to release is selected from a thermal mechanism, a mechanical mechanism, and an acoustic mechanism.

17. The device of claim 16, wherein the thermal mechanism heats the first area from ambient temperature to a temperature ranging from 200° C. to 600° C. within 500 msec.

18. The device of claim 16, wherein the thermal mechanism is selected from resistive heating, optical heating, and chemical heating.

19. The device of claim 1, further comprising a power source and a controller.

20. The device of claim 19, wherein the controller controls the time interval between subsequent actuations of the mechanism configured to release.

21. The device of claim 19, wherein the controller controls the amount of first compound released by the mechanism configured to release.

22. The device of claim 19, wherein the controller is adjustable by a patient.

23. The device of claim 1, further comprising an actuation mechanism.

24. The device of claim 1, further comprising a lock-out mechanism.

25. A device comprising:
a housing defining an airway;
a support configured to couple to the airway comprising at least one first area and at least one second area, wherein a first compound is disposed on the at least one first area, and a second compound is disposed on the at least one second area, and wherein the second compound can counteract the pharmacological effects of the first compound; and
a mechanism configured to release the first compound disposed on the at least one first area into the airway.

26. The device of claim 25, wherein the support comprises more than one first area.

27. The device of claim 25, wherein the support comprises more than one second area.

28. A device comprising:
a housing defining an airway;
more than one support configured to couple to the airway comprising at least one first area and at least one second area, wherein a first compound is disposed on the at least one first area, and a second compound is disposed on the at least one second area, and wherein the second compound can counteract the pharmacological effects of the first compound; and
a mechanism configured to release the first compound disposed on the at least one first area into the airway.

29. The device of claim 28, wherein the more than one support comprises more than one first area and more than one second area.

30. A device comprising:
a housing defining an airway;
a first support configured to couple to the airway comprising at least one first area, wherein a first compound is disposed on the at least one first area;
a second support comprising at least one second area, wherein a second compound is disposed on the at least one second area, and wherein the second compound can counteract the pharmacological effects of the first compound; and
a mechanism configured to release the first compound into the airway.

31. The device of claim 30, wherein the first support comprises more than one first area.

32. The device of claim 30, wherein the second support comprises more than one second area.

33. A device comprising:
a housing defining an airway;
more than one first support configured to couple to the airway comprising at least one first area, wherein a first compound is disposed on the at least one first area;
more than one second support comprising at least one second area, wherein a second compound is disposed on the at least one second area, and wherein the second compound can counteract the pharmacological effects of the first compound; and
a mechanism configured to release the first compound into the airway.

34. The device of claim 33, wherein the first support comprises more than one first area.

35. The device of claim 33, wherein the second support comprises more than one second area.

36. A device comprising:
a housing defining an airway;
a support configured to couple to the airway comprising more than one first areas and a second area, wherein a first compound is disposed on the more than one first areas, and a second compound is disposed on the second area, and wherein the second compound can counteract the pharmacological effects of the first compound; and
a mechanism configured to thermally vaporize the first compound disposed on at least one of the first areas into the airway.

37. The device of claim 36, wherein an aerosol comprising the first compound is formed when the first compound is thermally vaporized into the airway.

38. The device of claim 37, wherein the aerosol does not comprise the second compound.

39. The device of claim 37, wherein the aerosol comprises a therapeutically effective amount of the first compound.

40. The device of claim 37, wherein the aerosol exhibits an average mass median aerodynamic diameter ranging from 1 μm to 3 μm.

41. The device of claim 40, wherein the aerosol exhibits a geometric standard deviation of the log-normal particle size distribution of less than 3.

42. The device of claim 36, wherein at least a portion of at least one of the first areas is disposed within the airway.

43. The device of claim 36, wherein the support is thermally conductive.

44. The device of claim 36, wherein the support comprises stainless steel.

45. The device of claim 36, wherein the support comprises multiple layers.

46. The device of claim 36, wherein the support comprises at least one of a metal foil, a metal plate, a metal shell, and a metal disk.

47. The device of claim 36, wherein the first area comprises a thermally conductive material.

48. The device of claim 36, wherein the first compound comprises a pharmaceutical compound.

49. The device of claim 36, wherein the first compound comprises an opioid analgesic.

50. The device of claim 49, wherein the opioid analgesic compound is selected from at least one of the following: fentanyl, sufentanyl, remifentanyl, morphine, hydromorphone, oxymorphone, codeine, hydrocodone, oxycodone, meperidine, methadone, nalbuphine, buprenorphine, and buorphanol.

51. The device of claim 36, wherein the second compound comprises an antagonist of an opioid analgesic.

52. The device of claim 36, wherein the first compound comprises at least fentanyl and the second compound comprises at least one fentanyl antagonist compound.

53. The device of claim 52, wherein the at least one fentanyl antagonist compound is selected from naloxone and naltrexone.

54. The device of claim 36, wherein the first compound comprises a thin film.

55. The device of claim 54, wherein the thickness of the thin film is less than 20 μm.

56. The device of claim 36, wherein the first compound comprises an abusable substance.

57. The device of claim 36, wherein the first area exhibits properties that facilitate release of the first compound.

58. The device of claim 57, wherein the properties are selected from at least one of the following: thermal properties, optical properties, electrical properties, physical properties, and mechanical properties.

59. The device of claim 36, wherein the second area exhibits properties that inhibit release of the second compound under conditions in which the first compound is released.

60. The device of claim 59, wherein the properties are selected from at least one of the following: thermal properties, optical properties, electrical properties, physical properties, and mechanical properties.

61. The device of claim 36, wherein less than 5% of the first compound is degraded during thermal vaporization.

62. The device of claim 36, further comprising a power source and a controller.

63. The device of claim 62, wherein the controller controls the time interval between subsequent actuations of the mechanism configured to release.

64. The device of claim 62, wherein the controller controls the amount of first compound released by the mechanism configured to release.

65. The device of claim 62, wherein the amount of first compound released in a single actuation of the device is adjustable by a patient.

66. The device of claim 36, further comprising an actuation mechanism.

67. The device of claim 66, wherein the actuation mechanism comprises an airflow sensor.

68. The device of claim 67, wherein the actuation mechanism is activated when the velocity of the airflow through the airway exceeds a pre-established threshold.

69. The device of claim 68, wherein the pre-established threshold is at least 1 m/sec.

70. The device of claim 36, further comprising a lock-out mechanism.

71. The device of claim 70, wherein the lock-out mechanism controls the time interval between successive actuations of the mechanism configured to thermally vaporize.

72. The device of claim 36, wherein the mechanism configured to thermally vaporize is selected from resistive heating, optical heating, and chemical heating.

73. The device of claim 36, wherein an airflow is generated through the airway upon inhalation by a patient.

74. The device of claim 36, further comprising at least one airflow control valve operatively connected to the airway.

75. The device of claim 74, wherein the at least one airflow control valve is configured to maintain the airflow rate through the airway to range from 10 L/min to 120 L/min.

76. The device of claim 36, further comprising a mechanism configured to move the support.

77. A method of producing an aerosol of a first compound comprising:
providing at least one first area on which is disposed a first compound, and at least one second area on which is disposed a second compound, wherein the second compound can counteract the pharmacological effects of the first compound;
providing an airflow over at least a portion of the least one first area; and
releasing the first compound from at least a portion of the at least one first area into the airflow;
wherein the first compound forms an aerosol in the airflow.

78. A method of administering a therapeutically effective amount of a first compound to a patient comprising inhaling an aerosol produced by a device comprising:
a housing defining an airway;
at least one support configured to couple to the airway comprising at least one area selected from a first area and a second area, wherein a first compound is disposed on the first area, and a second compound is disposed on the second area, and wherein the second compound can counteract the pharmacological effects of the first compound; and
a mechanism configured to release at least a portion of the first compound into the airway;
wherein the device comprises at least one first area and at least one second area.

79. A method of administering a therapeutically effective amount of at least one first compound to a patient comprising inhaling an aerosol produced using the method comprising:
providing at least one first area on which is disposed a first compound, and at least one second area on which is disposed a second compound, wherein the second compound can counteract the pharmacological effects of the first compound;
providing an airflow over at least a portion of the least one first area; and
releasing the first compound from at least a portion of the at least one first area into the airflow;
wherein the first compound forms an aerosol in the airflow.

80. A method of treating a disease in a patient in need of such treatment comprising administering to the patient an aerosol comprising a therapeutically effective amount of at least one first compound, wherein the aerosol is produced by a device comprising:
a housing defining an airway;
at least one support configured to couple to the airway comprising at least one area selected from a first area and a second area, wherein a first compound is disposed on the first area, and a second compound is disposed on the second area, wherein the second compound can counteract the pharmacological effects of the first compound; and
a mechanism configured to release the first compound into the airway;
wherein the device comprises at least one first area and at least one second area.

81. A method of treating a disease in a patient in need of such treatment comprising administering to the patient an aerosol comprising a therapeutically effective amount of at least one first compound, wherein the aerosol is produced by the method comprising:
providing at least one first area on which is disposed a first compound, and at least one second area on which is disposed a second compound, wherein the second compound can counteract the pharmacological effects of the first compound;
providing an airflow over at least a portion of the least one first area; and
releasing the first compound from at least a portion of the at least one first area into the airflow;
wherein the first compound forms an aerosol in the airflow.

82. An aerosol produced using a device comprising:
a housing defining an airway;
at least one support configured to couple to the airway comprising at least one area selected from a first area and a second area, wherein a first compound is disposed on the first area, and a second compound is disposed on the second area, wherein the second compound can counteract the pharmacological effects of the first compound; and
a mechanism configured to release the first compound into the airway;
wherein the device comprises at least one first area and at least one second area.

83. An aerosol produced by the method of:
providing at least one first area on which is disposed a first compound, and at least one second area on which is disposed a second compound, wherein the second compound can counteract the pharmacological effects of the first compound;
providing an airflow over at least a portion of the least one first area; and
releasing the first compound from at least a portion of the at least one first area into the airflow;
wherein the first compound forms an aerosol in the airflow.
